(19) United States
(12) Patent Application Publication
(10) Pub. No.: US 2026/0317792 A1
(43) Pub. Date: Oct. 15, 2026

(54) PROCESS FOR PRODUCING A METHANE-ENRICHED GAS

(71) Applicant: Hitachi Zosen Inova Schmack GmbH, Schwandorf (DE)

(72) Inventor: Matthias SCHOLZ, Schwandorf (DE)

(73) Assignee: Hitachi Zosen Inova Schmack GmbH, Schwandorf (DE)

(21) Appl. No.: 19/482,669

(22) PCT Filed: Apr. 17, 2024
(86) PCT No.: PCT/EP2024/060366

§ 371 Date: Nov. 7, 2025

(30) Foreign Application Priority Data
May 10, 2023 (DE) 102023112274.6

(51) Int. Cl. C12P 5/02

(52) U.S. Cl. CPC C12P 5/023

(57) ABSTRACT

The invention relates to a process for generating a methane-enriched gas using a bioreactor. The bioreactor comprises: at least one inlet for supplying hydrogen and carbon dioxide into the bioreactor; at least one inlet for supplying fresh substrate into the bioreactor; at least one outlet for removing used substrate from the bioreactor; and at least one outlet for removing methane-enriched gas formed in the bioreactor. The process for generating a methane-enriched gas comprises the steps of: providing such a bioreactor; removing from the bioreactor the methane-enriched gas formed in the bioreactor; determining the amount of methane formed in the bioreactor in a predetermined period of time; removing used substrate from the bioreactor; and supplying fresh substrate into the bioreactor. The amount of fresh substrate supplied into the bioreactor depends on the amount of methane formed in the bioreactor in the predetermined period of time.

### CROSS-REFERENCE TO RELATED APPLICATIONS

This application claims the benefit of DE 10 2023 112 274.6 filed May 10, 2023 and PCT/EP2024/060366 filed Apr. 17, 2024, the disclosures of which are hereby incorporated herein in its entirety by reference.

### BACKGROUND OF THE INVENTION

### Technical Field

The invention relates to a process for producing a methane-enriched gas.

### Prior Art

In the context of changes to energy policies, the proportion of energy production taken up by renewable resources is increasing rapidly. Because directly renewable forms of energy such as power from wind or photovoltaics are not continuously and uniformly available either from a temporal or a quantitative standpoint, and in this regard, demand from consumers also fluctuates with time, storage possibilities for renewable energies are becoming increasingly important.

In this regard, methane is of fundamental importance as a chemical energy carrier because it has multiple potential applications in the areas of heat production, fuels and power production; it constitutes a form of energy which is readily stored and, in contrast to hydrogen, it can be used in existing infrastructures with the natural gas grid and natural gas filling stations.

Methane is produced from the starting materials carbon dioxide and hydrogen. In the vast majority of processes described in the prior art, methanation is carried out with a ratio of the educt gases H₂ to CO₂ of 4:1 so that, according to the reaction equation for the methanation reaction

4 H₂+CO₂→CH₄+2 H₂O,
there is no surplus educt gas. As a rule, in these systems, the hydrogen is provided by the electrolysis of water with the aid of an electrolyser, wherein surplus power or other cheap power is used. The carbon dioxide which is required for the production of methane can derive from various sources such as industrial or combustion waste gases, but preferably, climate-friendly CO₂ from renewable sources such as biomass is used, such as that which arises from biogas plants or also as digester gas in sewage treatment plants.

Particularly in energy conversion systems which, according to the “power to gas” principle, convert surplus power into the gaseous chemical energy carrier methane, a biological methanation is often carried out in which biomethane is formed by methanogenic microorganisms. In contrast to chemically catalysed methanation, for example using the Sabatier process, biological methanation is carried out without expensive and sensitive catalysts and makes fewer demands as regards reaction conditions such as temperature and pressure as well as the purity of the starting gases CO₂ and H₂.

In a reactor for biological methanation, in which the desired product methane is produced continuously with the aid of an aqueous medium, it is usual to exchange a certain proportion of medium from time to time so that a suitable concentration of essential minerals is permanently maintained and in addition, metabolites which could inhibit the methanogenesis are removed.

WO 2008/094282 A1 describes a biological system of this type for the production of methane from hydrogen and carbon dioxide using a microorganism culture in a culture medium. The carbon dioxide derives from an industrial process, while the hydrogen is obtained, inter alia, by electrolysis using cheap surplus power. Exchange of the medium therein is carried out at a constant rate, and so a constant volume of medium is exchanged per unit reaction period. EP 3 072 979 A1 and also WO 2014/076062 A1 describe similar processes and also carry out an exchange of medium at a constant rate.

Because the hydrogen for the biological methanation is formed by the electrolysis of water with the aid of an electrolyser and so the current used for it is available from wind power or photovoltaics but not in a constant quantity over long periods of time, in “power-to-gas” plants, significantly different quantities of methane are formed over the course of time. It has been shown that an exchange of medium at a constant rate as described in the prior art in a system of this type cannot guarantee a permanent, stable methane production with good gas quality.

There is therefore a further need for a process for biological methanation with the aid of which a methane-enriched gas with a good product gas quality can be produced using hydrogen and CO₂ in a stable process.

### SUMMARY

The objective of the invention is to provide a process for biological methanation which is suitable for the production of a methane-enriched gas with a good product gas quality in a stable process using hydrogen and CO₂.

In accordance with the invention, this objective is achieved by means of the process for the production of a methane-enriched gas and a plant for carrying out such a process. Further advantageous details, aspects and embodiments of the present invention are defined in the description.

The present invention provides a process for the production of a methane-enriched gas, comprising the steps of:
a) providing a bioreactor having
   at least one inlet for supplying hydrogen and carbon dioxide to the bioreactor,
   at least one inlet for supplying fresh substrate to the bioreactor,
   at least one outlet for removing old substrate from the bioreactor,
   at least one outlet for removing methane-enriched gas formed in the bioreactor,
b) removing the methane-enriched gas formed in the bioreactor from the bioreactor,
c) determining the quantity of methane formed in the bioreactor in a predetermined time period,
d) removing old substrate from the bioreactor, and
e) supplying fresh substrate to the bioreactor.

In accordance with the invention, the quantity of fresh substrate supplied to the bioreactor is a function of the quantity of methane formed in the bioreactor in the predetermined time period.

The process in accordance with the invention is carried out with the aid of a bioreactor for the production of a methane-enriched gas by means of biological methanation. The bioreactor may be configured in the form of different reactor types such as, for example, a continuous stirred-tank reactor, a column reactor, a tube reactor, a bubble column, a trickle bed reactor or also as a reactor cascade. It should be produced from a material which can withstand pressure and heat, for example stainless steel. Compared with the anaerobic fermenters of a biogas plant or a digestion tower of a sewage treatment plant, the reactor volume of a bioreactor for biological methanation is significantly smaller for the production of the same quantity of biomethane, for example in the range of 1/10 to 1/1000 of the volume of a fermenter of a biogas plant or a digestion tower of a sewage treatment plant.

The bioreactor is supplied with a carbon dioxide-containing gas as well as with an externally produced hydrogen-containing gas. The hydrogen required for the biological methanation is preferably produced by the electrolysis of water with the aid of an electrolyser. Preferably in this regard, the electrolyser is operated using power which is derived from renewable energy.

Particularly preferably, surplus power from the power grid is used. The provision of surplus power is usually overseen by a balancing energy group, so that the plant for biological methanation can participate in the power balancing market at the level of the corresponding electrolyser's output. Even if the electrolyser does not participate in the power balancing market, cheap power from other sources (for example from special power tariffs) could be used. Alternatively, hydrogen from other sources such as hydrolysis gas, synthesis gas, product gas from chemical reactions, H₂ of biological origin such as H₂ produced by algae or H₂ from photocatalysis, for example, can be used for the biological methanation.

In the process in accordance with the invention, the exchange of substrate in the bioreactor takes place in a manner such that the quantity of fresh substrate supplied to the bioreactor is a function of the quantity of methane formed in the bioreactor in the predetermined time period. The concentrations of the various salts and minerals in the bioreactor can be kept almost constant by the substrate exchange which is carried out as a function of the methane which is formed, whereupon a stable methanation process is guaranteed.

Thus, the methane formation rate which is obtained in the respective biological methanation process can serve as the parameter for the frequency of the exchange of a specific quantity of substrate or as a parameter for the quantity of exchanged substrate. At high methane formation rates, the substrate exchange takes place significantly more frequently or in larger quantities than at lower methane formation rates.

With the exception of the initial filling upon start-up of the bioreactor, the exchange of the substrate is carried out as a function of the quantity of biomethane produced during the ongoing operation of the reactor. For the exchange, old substrate is cyclically discharged and the reactor is subsequently replenished with fresh substrate. Here, the water originating from the methanation reaction is also withdrawn with the old substrate. In the simplest case, when the substrate is based on digested sludge or fermentation residue, the substrate is returned to the sewage treatment plant or biogas plant.

The rate at which the substrate in the bioreactor is exchanged is not set as a function of the quantity of methane formed as the reaction product in any of the processes for biological methanation which are known in the prior art. In known cases, the exchange rate [h⁻¹] is related to the reaction period for the methanation, wherein as a rule, the proportion of the reactor volume which is exchanged per unit of time is specified. The feed rate [1 h⁻¹] information is also related to the reaction period for methanation. Here, the volume of the reactor contents is taken into account and the volume of substrate per unit time to be added is specified. In a few cases, the rate of substrate exchange is also related to the doubling time of the methanogenic microorganisms which are responsible for the methane formation, wherein the exchange of the substrate is carried out at a rate which is a reciprocal of the doubling time.

A decisive advantage of the process in accordance with the invention compared with other known processes of the prior art is that the economic efficiency is increased because the methane formation rate is significantly increased. At the same time, in order to introduce the process in accordance with the invention into existing plants, practically no investment is required because pre-existing infrastructure and technology can be employed.

The bioreactor contains an aqueous reaction medium which serves as a substrate or nutrient medium for the methanogenic microorganisms and therefore functions as a methanation medium. Either a synthetic culture medium for methanogenic microorganisms as described in the prior art (for example WO 2008/094282 A1, EP 2 675 904 B1) or a complex nutrient medium based on biomass, which substantially consists of a fermentation residue as the substrate and which may optionally be supplemented with nutrients and/or trace elements (for example EP 2 982 740 A1), is used as the medium in which the methanation reaction takes place.

The more nutrients and trace elements that are already contained in a fresh substrate, the fewer auxiliary materials need to be added. The fermentation residue from a biogas plant, in particular the fermentation residue from a later fermentation stage or from a secondary fermenter, may be used as the substrate, wherein the fermentation residue may also originate from a repository. In addition, in principle, any type of raw sludge and/or digested sludge from a sewage treatment plant may be suitable as a substrate for the bioreactor for biological methanation.

Methanogenic microorganisms, in particular hydrogenotrophic methanogenic microorganisms, are added to a synthetic culture medium in the form of a pure culture or a mixed culture of suitable microorganisms. As a rule, when a fermentation residue is used as the substrate, methanogenic microorganisms are already present, however optionally, additional methanogenic microorganisms may be added, in particular hydrogenotrophic methanogenic microorganisms. Thermophilic methanogens of the genus Methanothermobacter are particularly suitable.

In accordance with a particularly preferred embodiment of the present invention, the quantity of fresh substrate, m_{fresh substrate}, supplied to the bioreactor is a function of the quantity of methane, Vₘₑₜₕₐₙₑ, formed in the bioreactor in the predetermined time period and an exchange rate, EXR, and satisfies the equation m_{fresh substrate}=Vₘₑₜₕₐₙₑ·EXR, wherein the exchange rate EXR satisfies the condition EXR>0. This embodiment enables a defined exchange rate to be specified for the process for biological methanation. A stable methanation process with good product gas quality is obtained.

Preferably, the quantity of fresh substrate, m_{fresh substrate} [kg], supplied to the bioreactor is a function of the quantity of methane, Vₘₑₜₕₐₙₑ [Nm³], formed in the bioreactor in the predetermined time period and of an exchange rate, EXR [kg/Nm³], and satisfies the equation m_{fresh substrate} [kg]=Vₘₑₜₕₐₙₑ [Nm³]·EXR [kg/Nm³], wherein the exchange rate EXR satisfies the condition 0.01 kg/Nm³≤EXR≤10 kg/Nm³.

Particularly preferably, the exchange rate EXR satisfies the condition 1 kg/Nm³≤EXR≤6 kg/Nm³. Thus, for example, an exchange rate of 4 kg per Nm³ of methane formed in the bioreactor can be specified. This means that 4 kg of fresh substrate is added to the bioreactor as soon as one standard cubic metre of pure methane has been formed.

It will be clear to the person skilled in the art that for a known density of the substrates, all of the quantities by weight can be replaced by volumetric quantities. The unit for the exchange rate can therefore, for example, be specified in kilograms of fresh substrate per cubic metre of methane formed or in litres of fresh substrate per cubic metre of methane formed.

It should be noted that in the context of the present text, the expression “quantity of methane formed in the bioreactor in a predetermined time period” should in fact be understood to mean exactly the quantity of fresh methane formed in the bioreactor in a predetermined time period. If the educt gas for the methanation reaction already contains methane because, for example, the carbon dioxide source used is crude biogas or fermentation gas, then this must be taken into account when analysing the measurement results, which reflect the quantity of methane in the product gas. When calculating the quantity of substrate to be exchanged, only the quantity of fresh methane formed in the bioreactor in a predetermined time period has to be taken into consideration.

Preferably, the fresh substrate supplied in step e) of the process in accordance with the invention is a biological substrate with an increased viscosity or a culture medium. Particularly preferably, the biological substrate with an increased viscosity is fermentation residue from a biogas plant or sewage sludge. Methanation reactors are often constructed in the vicinity of biogas plants or sewage treatment plants, bringing particular advantages having regard to the availability of fresh substrate and the low costs associated therewith.

In accordance with a preferred embodiment, the culture medium contains one or more of: microorganisms, nutrients, and trace elements. In accordance with one embodiment, nutrients and/or trace elements are also supplied to the bioreactor as a function of the quantity of biomethane which is formed. A stable methanation process with an excellent product gas quality is established.

In the processes which are known in the prior art, the addition of nutrients is carried out as a function of the requirements of the cultured cells and the consumption of nutrients in the bioreactor. An addition of nutrients as a function of the quantity of methane which is formed has not been described in the prior art.

Particularly preferably, after step d) and before, together with or after step e), the step f) of supplying one or more auxiliary substances to the bioreactor is carried out, wherein the one or more auxiliary substances are nutrients, trace elements, defoaming agents, acids, bases or mixtures thereof. As already mentioned, by carrying out the substrate exchange as a function of the quantity of methane formed, the salt and mineral concentration in the bioreactor can be kept almost constant, whereupon a stable methanation process is guaranteed. In a particularly advantageous manner, the salt and mineral concentrations can be kept constant in the bioreactor if at the same time an appropriate addition of nutrients and trace elements is carried out, which in addition prevents both a deficiency and an overdose of nutrients and trace elements.

In principle, the quantity of added auxiliary substances such as acids or bases for adjusting the pH, defoaming agents, trace element solutions or nutrient solutions, for example, influences the volume of the reactor contents, and so does sampling, which leads to a reduction in the reactor contents. However, as a rule, all of the auxiliary substances are added in such a concentrated form that they only make up a fraction of the reactor contents and therefore can be disregarded in the volume considerations.

The removal of old substrate from the bioreactor and the supply of fresh substrate to the bioreactor can in principle be carried out continuously or also discontinuously in a batchwise substrate exchange at intermittent intervals.

A “continuous substrate exchange” advantageously means that an almost-constant substrate fill level can be maintained in the bioreactor. Even the volumetric gas flows remain substantially constant for a specified methane formation rate. Compared with a batchwise substrate exchange, the pumps which deliver fresh substrate and/or withdraw spent substrate can have smaller dimensions because they continuously exchange a small quantity of substrate. The steps d) and e) of the process in accordance with an embodiment of the invention are therefore preferably carried out continuously.

Particularly preferably, the removal of old substrate from the bioreactor in step d) and the supply of fresh substrate to the bioreactor in step e) are carried out in a manner such that the total quantity of substrate present in the bioreactor remains substantially constant, wherein the total quantity of substrate present in the bioreactor corresponds to a predetermined target quantity of substrate.

In the case of a batchwise substrate exchange, firstly, old substrate is discharged and afterwards, fresh substrate is added until a predetermined target value is reached for the set reactor volume. A batchwise substrate exchange has the advantage that the substrate pumps used for the substrate exchange do not have to run continuously, whereupon less wear occurs. The steps d) and e) of the process in accordance with an embodiment of the invention are therefore preferably carried out in a batchwise manner.

Independently of the type of substrate (digested sludge, fermentation residue, synthetic medium), the pressure in the reactor is kept constant. This means that during the substrate exchange, significant variations in the volumetric flow of the product gas occurs, which become smaller during the discharge of old substrate and larger when substrate is added. For this reason, a downstream gas preparation plant must be designed for a higher volumetric flow of gas than would be appropriate for the volumetric flow of gas specified by the respective volumetric flow of educt gas. In the case of a batchwise substrate exchange, this effect is considerably more pronounced than in the case of a continuous substrate exchange, because an increased quantity of substrate is exchanged per unit of time during batchwise exchange.

Particularly preferably, the quantity m_{fresh substrate} [kg] of fresh substrate supplied to the bioreactor per batch is a function of a predetermined target quantity of substrate, m_{target substrate} [kg], present in the bioreactor and satisfies the condition 0.01 m_{target substrate}≤m_{fresh substrate}≤0.1 m_{target substrate}. The quantity m_{fresh substrate} [kg] of fresh substrate supplied to the bioreactor can be calculated from knowing the quantity of substrate contained in the respective bioreactor. A practical solution has been shown to be to carry out a substrate exchange when approximately 5% of the substrate volume is to be exchanged. Up to 10% exchange of the substrate volume can also be tolerated without the variations in the fill level of the reactor having too great an effect on the biological methanation process.

Because, according to the equation

CO₂+4 H₂+→CH₄+2 H₂O,
water is also formed in addition to methane, the quantity of the methane formed in the bioreactor in a predetermined time period can be determined by determining the quantity of water formed in the bioreactor in the predetermined time period. In accordance with a further preferred embodiment of the present invention, therefore, the determination of the quantity of methane, Vₘₑₜₕₐₙₑ, formed in the bioreactor in a predetermined time period in step c) is carried out by determining the quantity of water, V_{water}, formed in the bioreactor in the predetermined time period. Under standard conditions (atmospheric pressure, 0° C.), 1.6 litres of water are formed per standard cubic metre of methane. A quantity of 1 Nm³ of methane which is formed in this case therefore corresponds to a quantity of 1.6 L of water. The formation of water also explains why as a rule, the quantity of old substrate discharged from the reactor is not identical to the quantity of fresh substrate which is added to the reactor. In the case of the presence of carbon monoxide in the educt gas, the methane formation can also occur in accordance with the equation

3 H₂+CO→CH₄+H₂O

As a rule, however, the quantities of carbon monoxide in the educt gas are so small that this effect can be ignored.

The present invention also concerns a plant for carrying out one of the processes described above, wherein the plant has at least one bioreactor. The bioreactor has at least one inlet for supplying hydrogen and carbon dioxide to the bioreactor, at least one inlet for supplying fresh substrate to the bioreactor, at least one outlet for removing old substrate from the bioreactor and at least one outlet for removing methane-enriched gas formed in the bioreactor.

The plant in accordance with the invention for carrying out one of the processes described above furthermore comprises a device for determining the quantity of methane formed in the bioreactor in a predetermined time period and a computer unit, wherein the computer unit is designed and configured to calculate the quantity of fresh substrate to be supplied to the bioreactor as a function of the quantity of methane formed in the bioreactor in the predetermined time period.

The bioreactor of the plant in accordance with the invention is fed with a carbon dioxide-containing gas as well as with an externally produced hydrogen-containing gas. The hydrogen required for the biological methanation is preferably produced by means of an electrolyser. Preferably, the hydrogen is produced in the electrolyser by the electrolysis of water using surplus power from the power grid. The provision of surplus power is usually overseen by a balancing energy group, so that the plant for biological methanation can participate in the power balancing market at the level of the corresponding electrolyser's output. Even when the electrolyser does not participate in the power balancing market, cheap power from other sources (for example from special power tariffs) could be used. Alternatively, hydrogen from other sources such as hydrolysis gas, synthesis gas, product gas from chemical reactions, H₂ of biological origin, such as H₂ produced by algae or H₂ from photocatalysis, for example, may be used for the biological methanation.

The bioreactor for biological methanation advantageously has at least one inlet for supplying hydrogen and carbon dioxide to the bioreactor. This is advantageously a suitable system for introducing the CO₂-containing gas as well as the hydrogen-containing gas into the liquid reactor medium. The CO₂-containing gas as well as the hydrogen-containing gas are preferably introduced into the bioreactor via supply lines. Preferably, the two gases have already been mixed in a gas mixing chamber prior to introduction into the reactor and are subsequently fed into the reactor via a common educt gas line. The H₂-containing and CO₂-containing educt gases are supplied directly into the bioreactor, for example via at least one supply line, preferably into the lower region of the bioreactor.

The reactor for biological methanation has at least one outlet for removing methane-enriched gas formed in the bioreactor. The methane-enriched gas which is formed is removed from the bioreactor via a product gas line which is preferably disposed at the upper end of the bioreactor.

As a rule, prior to any further upgrading of the product gas, a gas treatment with the steps of gas drying and gas cooling as well as optional desulphurization, gas scrubbing in order to separate further components such as ammonia, for example, and/or H₂ separation and/or CO₂ separation are carried out. In this manner, a prepared SNG (Substitute Natural Gas) or bionatural gas or biomethane which can be fed into a public gas distribution system can be prepared from the methane-enriched gas leaving the bioreactor as the product gas. In addition to feeding SNG into the natural gas distribution system, it is possible to use it as fuel, for example in the form of a bionatural gas filling station or a material use. A further upgrading possibility is the re-conversion of biomethane with simultaneous heat production, for example via a combined heat and power unit, during times when the electricity supply is too low. In this case, temporary storage is required for the biomethane produced. This and also a combined heat and power unit are often already integrated into the corresponding plants and therefore can advantageously be used for the process in accordance with the invention.

In addition, the bioreactor is advantageously equipped with a control valve for regulating the pressure in the bioreactor and corresponding pressure-stable components, so that a reaction pressure which is higher than the atmospheric pressure, in particular an over-pressure of up to 30 bar, preferably an over-pressure of up to 16 bar, can be set. The bioreactor is optionally equipped with sensors for the measurement of temperature, pressure and pH. In addition, a system for adjusting the pH or for adding base or acid may be provided.

In order to control the substrate exchange technically, the quality of the product gas is determined after leaving the methanation reactor. As a rule, devices for measuring the CO₂ content of the product gas as well as the CH₄ content and the H₂ content of the product gas are already present in a methanation system and can therefore be used to advantage. Preferably, the product gas analysis device directly follows the methanation reactor or is located in the gas preparation zone. In this manner, the quantity of methane formed in the bioreactor is measured continuously in the methane-enriched gas removed from the bioreactor and can be determined for any respectively predetermined time periods.

Subsequently, the computer unit provided in the plant in accordance with the invention can calculate the quantity of fresh substrate to be supplied to the bioreactor as a function of the quantity of methane formed in the bioreactor in the predetermined time period from the measured values obtained in this manner.

As an example, a suitable computer may be used as the computer unit which, of course, does not necessarily have to be located directly at the methanation reactor, but can also be connected to it via lines or be controlled remotely. Advantageously, the measured values obtained from the device for determining the quantity of methane formed in the bioreactor in a predetermined time period are transferred to the computer unit via lines or wirelessly.

Preferably, the computer unit is a programmable logic controller (PLC) which, particularly preferably, is provided directly at the methanation plant. The measured values are transferred to the PLC, which then calculates the quantity of fresh substrate to be supplied to the bioreactor as a function of the quantity of methane formed in the bioreactor in the predetermined time period. Thus, the PLC calculates when a change of substrate is due and triggers it by controlling specific actuators such as a substrate pump, for example.

Particularly preferably, the computer unit carries out a calculation of the quantity of fresh substrate, m_{fresh substrate}, to be supplied to the bioreactor as a function of the quantity of methane, Vₘₑₜₕₐₙₑ, formed in the bioreactor and an exchange rate, EXR, in accordance with the equation m_{fresh substrate}=Vₘₑₜₕₐₙₑ·EXR, wherein the exchange rate EXR satisfies the condition EXR>0. This embodiment means that a defined exchange rate can be specified for the process for biological methanation. A stable methanation process with good product gas quality is obtained.

Preferably, by means of the computer unit, the quantity of fresh substrate, m_{fresh substrate} [kg], to be supplied to the bioreactor is calculated as a function of the quantity of methane, Vₘₑₜₕₐₙₑ [Nm³], formed in the bioreactor in the predetermined time period and the exchange rate EXR [kg/Nm³] in accordance with the equation m_{fresh substrate} [kg]=Vₘₑₜₕₐₙₑ [Nm³]·EXR [kg/Nm³], wherein the exchange rate EXR satisfies the condition 0.01 kg/Nm³≤EXR≤10 kg/Nm³, preferably the condition 1 kg/Nm³≤EXR≤6 kg/Nm³. Thus, for example, an exchange rate of 4 kg of substrate per Nm³ of methane formed in the bioreactor can be specified. This means that 4 kg of fresh substrate is added to the bioreactor as soon as one standard cubic metre of pure methane has been formed.

Particularly preferably, the plant furthermore has at least one substrate pump, wherein the at least one substrate pump is designed and configured to deliver fresh substrate to the inlet for supplying fresh substrate to the bioreactor and/or to remove old substrate from the bioreactor. A substrate pump constitutes the simplest and most failure-resistant device for the technical accomplishment of the substrate exchange.

In accordance with a particularly preferred embodiment, the computer unit is designed and configured to control the at least one substrate pump, wherein the control of the at least one substrate pump is carried out in a manner such that fresh substrate is supplied to the bioreactor in accordance with the equation m_{fresh substrate}=Vₘₑₜₕₐₙₑ·EXR. This embodiment enables the bioreactor to be operated fully automatically with continuous or discontinuous substrate exchange.

Particularly preferably, the computer unit is designed and configured to control the at least one substrate pump, wherein the control of the at least one substrate pump is carried out in a manner such that fresh substrate is continuously supplied to the bioreactor in a quantity, m_{fresh substrate} [kg], such that the total quantity, m_{total substrate} [kg], of substrate present in the bioreactor remains substantially constant, wherein the total quantity of substrate present in the bioreactor corresponds to a predetermined target quantity of substrate, m_{target substrate} [kg].

### Definitions

Biogas: The term “biogas” should be understood to mean a gas which is formed in an anaerobic fermentation in a plant for the production of biogas under the action of various microorganisms. It contains methane and carbon dioxide as the main components. In addition, it contains water vapour and possibly small proportions of hydrogen, nitrogen, oxygen, hydrogen sulphide and ammonia. This type of biogas is also termed crude biogas and can, for example, be supplied to a combined heat and power unit in order to produce power and heat. If the crude biogas is processed further in order, for example, to be subsequently fed into a natural gas distribution system, then carbon dioxide, inter alia, is separated out. The separated carbon dioxide-rich gas from a biogas process can be used as the carbon dioxide-containing educt gas for the biological methanation. However, crude biogas is also suitable as the carbon dioxide-containing educt gas for the biological methanation. A plant for the production of biogas can, for example, be a biogas plant in which the biogas is produced from biomass. Furthermore, the plant for the production of biogas can also be a sewage treatment plant which has a digestion tower. The biogas produced in a sewage treatment plant is also known as digester gas or sewer gas.

Educt gas: Educt gases for methanation are hydrogen-containing gases and carbon dioxide-containing gases or hydrogen and carbon dioxide, respectively.

Methane-enriched gas=product gas=biomethane: A “methane-enriched gas” should be understood to mean a gas which is formed by the action of hydrogenotrophic methanogenic microorganisms in an anaerobic bioreactor with hydrogen-containing and carbon dioxide-containing gas being supplied. At the same time, the methane-enriched gas is the product gas which is formed from the educt gases in the bioreactor in the biological methanation process and which leaves the bioreactor via the product gas line. In addition to CH₄, the methane-enriched gas can also contain proportions of the educt gases introduced into the bioreactor for methanation, for example unreacted hydrogen and carbon dioxide or other gases which are present in smaller quantities, such as nitrogen, hydrogen sulphide or ammonia, as they occur in a crude biogas, for example, so that the methane-enriched gas does not have to consist of 100% methane. However, methane is the main component of the methane-enriched gas. The methane-enriched gas which is produced in the context of the present application is also a biomethane and can also be considered to be the methane-enriched biogas when a crude biogas is used as the carbon dioxide-containing gas. The term “biomethane” should be understood to be distinguished from synthetic methane, which is formed in chemically catalysed methanation.

Bionatural gas and SNG (substitute natural gas): “Bionatural gas” should be understood to mean a methane gas which can be fed into the natural gas distribution system in accordance with the current guidelines (in Germany, for example, DVGW Guidelines G260, G262) and which has been produced in a biological methanation using CO₂ of a biogenic origin. The term “SNG”, the abbreviation for “synthetic natural gas”, is substantially synonymous with this, i.e. a technically produced substitute for natural gas which can be used in the same manner and which can be fed into the natural gas distribution system as an exchange gas.

Biomass: The term “biomass” as used in the context of the invention should be understood to mean all types of fermentable sustainable crude materials such as corn, grain, grass, silphium, sugar beets, but also animal excrement such as cattle or pig manure, horse manure or dry chicken manure, as well as municipal or industrial waste water along with biological waste or organic waste from food production or food processing, as well as any mixtures of these fermentation substrates.

Fermentation residue: The term “fermentation residue” or “digestate” describes the residue of fermentation substrate which is left behind following fermentation of biomass in a plant for the production of biogas. The term “fermentation residue” should be understood to mean, for example, the residue after the final temperature-controlled fermentation stage (for example secondary fermenter) in a biogas plant. Similarly, the term “fermentation residue” should be understood to include digested sludge from sewage treatment plant digestion towers.

Bioreactor=methanation reactor: A reactor with a methanation medium including methanogenic microorganisms in which the product gas methane is formed from carbon dioxide and hydrogen.

Methanation: The term “methanation” should be understood to mean methane formation starting from the gaseous substances hydrogen and carbon dioxide as educt gases. “Biological methanation” describes the formation of biomethane with the aid of hydrogenotrophic methanogenic microorganisms in an aqueous medium. It essentially occurs in accordance with the chemical equation: 4H₂+CO₂→CH₄+2H₂O. If carbon monoxide is contained in one of the educt gases, the methane can additionally be formed in accordance with the equation: 3H₂+CO→CH₄+H₂O. Other ways of forming methane, for example from acetate or methyl compounds, are not excluded, depending on the methanation medium employed and the reaction conditions used, but they play a subordinate role because carbon dioxide-containing gas and hydrogen-containing gas is added in overwhelming quantities.

Methanation medium: This describes the contents of the bioreactor reactor which is suitable for carrying out a biological methanation. The methanation medium is an aqueous reaction medium and constitutes the nutrient medium for the methanogenic microorganisms. It contains all of the nutrients and trace elements and other components which are necessary for the growth of the corresponding hydrogenotrophic methanogenic microorganisms. Either a synthetic culture medium for methanogenic microorganisms is used, or a complex nutrient medium based on biomass is used as the methanation unit; it substantially consists of a fermentation residue as the substrate, possibly supplemented with nutrients and/or trace elements. The composition of the methanation medium varies during methanation. On the one hand, during the reaction of carbon dioxide and hydrogen to form methane, water is also formed. In addition, during methanation, the addition of acids, bases and/or defoaming agents is necessary. During bacterial growth, some materials in the methanation medium are consumed, and others are formed. These procedures are the reason why more and more spent old substrate has to be changed for fresh substrate during the methanation process.

Substrate: The substrate can be a biological substrate with an increased viscosity (for example fermentation residue, sewage sludge) or a culture medium for methanogenic microorganisms. The substrate itself can already be sufficient for the biological methanation, but it can also be supplemented with other auxiliary substances.

Fresh substrate: Material which is added to the bioreactor and therefore forms a component of a methanation medium is described as fresh substrate.

Old substrate: Material in the bioreactor in which a methanation reaction has already taken place over a specific time period is described as old substrate. Old substrate is removed from the bioreactor at given times.

Auxiliary substances: Auxiliary substances supplement the substrate in the bioreactor. They may be nutrients, trace elements, microorganisms, acids, bases and defoaming agents. The auxiliary substances constitute an essential part of the substrate. They can already be contained in a biological substrate or in a culture medium in optimal quantities for the methanation. In this case, only fresh substrate is added to the bioreactor. Frequently, nutrients and/or trace elements are added as auxiliary substances via separate metering stations which contain concentrated solutions of these substances. As a rule, this is brought about via suitable pumps. The addition of the nutrients and/or trace elements is then carried out separately prior to adding the fresh substrate, but is quantitatively coupled to the quantity of fresh substrate which is added. Microorganisms are also added separately as auxiliary substances for the methanation, in particular methanogenic microorganisms, in particular hydrogenotrophic methanogenic microorganisms. An addition of microorganisms is preferably carried out with an inoculum which is added once at the start of a methanation process. As a rule, the addition of acids, bases as well as defoaming agents is carried out via individual metering stations with appropriate pumps, separately from the addition of the fresh substrate.

### BRIEF DESCRIPTION OF THE DRAWINGS

The invention will now be described in more detail in connection with the drawings and with the aid of exemplary embodiments. Obviously, the statements made with respect to the exemplary embodiments do not limit the invention. In the figures:

FIG. 1 shows, as a graphical representation, measured values for the product gas composition and procedural parameters when implementing an embodiment of the process in accordance with the present invention with sewage sludge as the fresh substrate at a constant exchange rate and different methane formation rates;

FIG. 2 shows, as a graphical representation, measured values for the product gas composition and procedural parameters when implementing an embodiment of the process in accordance with the present invention with sewage sludge as the fresh substrate at a constant methane formation rate and different exchange rates;

FIGS. 3A, 3B show, as a graphical representation, measured values for the product gas composition and procedural parameters when implementing an embodiment of the process in accordance with the present invention with a synthetic culture medium at a constant exchange rate and different methane formation rates, wherein the composition of the synthetic culture medium in the test forming the basis of the measured values of FIG. 3A differ from the composition of the synthetic culture medium in the test forming the basis of the measured values of FIG. 3B;

FIGS. 4A, 4B show, as a graphical representation, measured values for the product gas composition and procedural parameters when implementing a biological methanation in accordance with a known process from the prior art with sewage sludge as the fresh substrate at constant exchange times independently of the methane formation rate.

### DESCRIPTION OF EXEMPLARY EMBODIMENTS

Descriptions of preferred embodiments are disclosed in the examples provided herein.

### Example 1

A bioreactor with a volume of 80 kg of substrate equipped with a gassing agitator was operated with digested sludge from a sewage treatment plant. The specified exchange rate was 4 kg of substrate per Nm³ of methane formed. This means that 4kilograms of fresh substrate was added as soon as one standard cubic metre of pure methane had been formed.

During the methane formation in the bioreactor, the quantity of product gas formed was measured continuously. In connection with an analysis of the respective product gas quality, the quantity of methane formed was therefore determined via the methane content in the product gas.

Because the substrate exchange was carried out batchwise, a quantity of new substrate which was to be exchanged per substrate exchange in the bioreactor was specified. This can be described as the target mass for the fresh substrate. The target mass for the fresh substrate was 5 percent measured at the target value for the bioreactor contents. The bioreactor used on the pilot scale had a target value for the substrate mass of 80 kilograms. The lower limit for the substrate mass was 76 kilograms for an exchange of 5 percent of the bioreactor mass, resulting in a target mass for the fresh substrate of 4 kilograms per exchange.

For the stable operation of the methanation reactor and in order to optimise the methane formation rate, a supply of auxiliary substances was provided. These were defoaming agents, optional bases or acids in order to adjust the pH, as well as nutrients and trace elements. An individual metering station with an individual line into the bioreactor was provided for each of the auxiliary substances. In this regard, the metering stations were designed having regard to the expected consumptions and the amount of storage which was available.

Because the exchange rate was specified to be 4 kg/Nm³, the substrate exchange was triggered as soon as one standard cubic metre of methane had been formed since the last substrate exchange. For the substrate exchange, firstly, the bioreactor contents was discharged to the lower mass limit of 76 kilograms for the substrate mass in the bioreactor. The quantity of the discharged substrate here was more than 4 kilograms, because during the formation of one standard cubic metre of methane, a corresponding quantity of 1.6 L of water was formed. Subsequently, 4 kilograms of fresh substrate was pumped into the bioreactor via a substrate pump, so that the substrate mass in the bioreactor regained the target value of 80 kilograms.

For each substrate exchange, auxiliary substances corresponding to previously specified target values were supplied with the fresh substrate. Adding auxiliary substances such as nutrients and/or trace elements ensured that these materials were always available to the methanogenic microorganisms in sufficient quantities. The supply of these auxiliary substances was also carried out as a function of the quantity of methane formed, whereupon a suitable supply of nutrients and trace elements to the methanogens was ensured, independently of the bioreactor size.

### Example 2

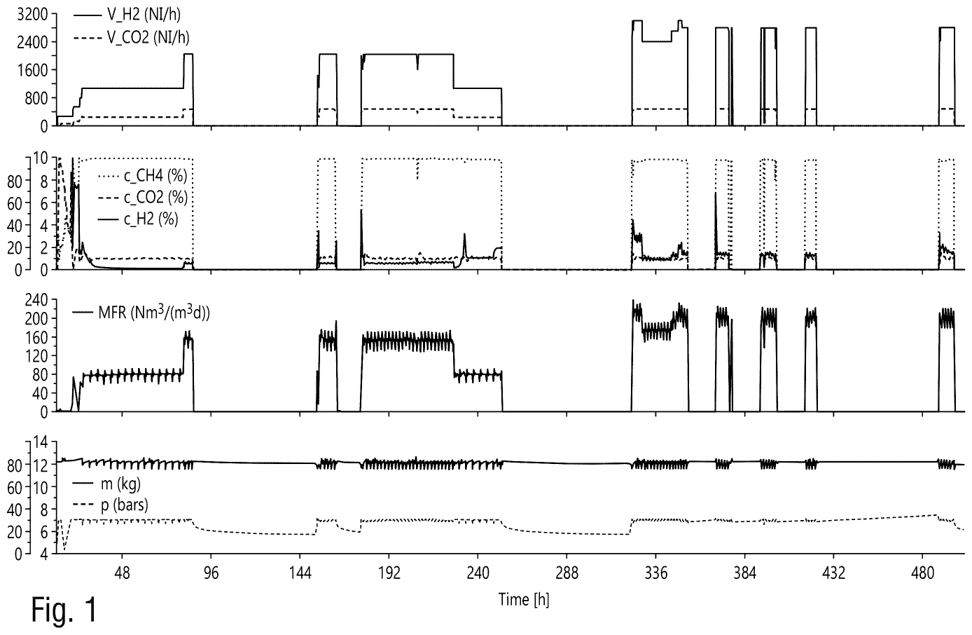

FIG. 1 shows, as a graphical representation as a function of the test time [h] and from top to bottom
measured values for the volumetric educt gas flows of hydrogen and carbon dioxide in standard litres per hour,
measured values for the product gas composition as the volumetric proportions of methane, carbon dioxide and hydrogen, given as a percentage of the product gas,
measured values for the methane formation rates, given in standard cubic metres of methane produced per cubic metre of reactor volume and per day, and
measured values for the fill quantity in the bioreactor [kg] and for the pressure in the reactor in bars.

The biological methanation was carried out in a bioreactor which contained digested sludge from a municipal sewage treatment plant as the methanation medium, to which individual trace elements and nutrients had been added. The biological methanation was carried out without adding a methanogenic microorganism. The production of the methane-enriched gas was carried out in a continuous stirred-tank reactor with a 80 litre reactor content, at a temperature of 65° C. and a pressure of 7 bar.

The addition of the educt gases H₂ and CO₂ was carried out from gas bundles each with pure H₂ and CO₂ in pressurized gas cylinders. The respective volumetric gas flow in standard litres per hour [Nl/h] was adjusted using a mass flow regulator from Bronkhorst. The volumetric flow of hydrogen (V_H2) was specified depending on the intended methane formation rate. The exact value for the supplied volumetric flow of carbon dioxide (V_CO2) was obtained after measuring the actual value for carbon dioxide and calculating the control deviation from the manipulated variable which was output by the CO₂ controller in connection with the specified volumetric flow of hydrogen.

The biological methanation was carried out over the entire time period at an exchange rate (EXR) of 4 kilograms of fresh substrate per standard cubic metre of methane formed [kg/Nm³]. The exchange of fresh substrate was carried out batchwise as described in Example 1. At a constant methane formation rate and at regular intervals, a portion of the reactor contents was discharged and subsequently replenished with fresh substrate or methanation medium, so that a certain amount of exchange was obtained with fresh material. This can be seen in the bottom diagram in FIG. 1 as small downward deflections and a subsequent increase in the mass.

Individual nutrients and trace elements were supplied to the reactor after the substrate exchange via a pump from a reservoir of concentrated stock solutions, respectively over a short time period. The respective quantity was in agreement with the quantity of exchanged substrate and was calculated according to predetermined target values for the individual nutrients and trace elements.

The bioreactor was started up with an appropriate metering of the volumetric educt gas flows of carbon dioxide and hydrogen over a time period of approximately 12 hours to a methane formation rate (MFR) of 80 standard cubic metres of methane per cubic metre of reactor contents per day [Nm³/(m³d)] and subsequently operated for 54 hours at this MFR. A methane-enriched gas with a quality of more than 98% methane, 1% carbon dioxide and less than 1% hydrogen was obtained. The gas quality of the product gas is shown in the second diagram from the top. The values for CH₄ can be read from the left hand scale on the Y-axis; the values for CO₂ and H₂ can be read from the right hand scale of the Y-axis.

In the bottom diagram, it can be seen from the measured values for the fill level in the bioreactor (left hand scale) that for a MFR of 80 Nm³/(m³d), a substrate exchange of 4 kg of old substrate for fresh substrate was carried out every 3.75 hours, corresponding to 5% of the fill level of the bioreactor. This was associated with pressure fluctuations in the bioreactor which can be seen in the measured values for the internal reactor pressure (right hand scale).

Directly thereafter, the bioreactor was set to a MFR of approximately 155 Nm³/(m³d) via the volumetric educt gas flows for 4 hours, before the educt gas supply was suspended. This increase was possible without problems. It can be directly read from the measured values for the reactor fill level that the substrate exchange was carried out every 2 hours, because the exchange rate is a function of the quantity of methane formed.

In the time period of what is known as “cold standby”, the bioreactor was not stirred and not heated for 64 hours, so that the operating temperature for the methanation of 65° C. dropped to the ambient temperature of approximately 25° C. This can be discerned by the fact that the pressure in the bioreactor dropped. There was no supply of substrate, nor of auxiliary substances. The biological methanation could be recommenced directly with a MFR of 155 Nm³/(m³d) with a short heating phase. After 8 hours of operation at a MFR of 155 Nm³/(m³d), a new “cold standby” was carried out for 12 hours, followed by a long methanation phase at a MFR of 155 Nm³/(m³d) over 48 hours, after which the MFR was returned to 80 Nm³/(m³d).

After a new “cold standby” phase of over 69 hours, the bioreactor was operated over a time period of 30 hours in total with methane formation rates of 210, 175 and 200 Nm³/(m³d). The quality of the gas was slightly poorer only directly after the new startup of the biological methanation after a long downtime, with approximately 3% hydrogen and approximately 96% methane.

Following this, what is known as a “hot standby” was carried out three times for 12 to 14 hours, each time interrupted for a methanation phase with a methane formation rate of 200 Nm³/(m³d), followed by a “hot standby” for 65 hours followed by an 8-hour methanation phase with a methane formation rate of 200 Nm³/(m³d). In the case of a “hot standby”, the bioreactor continued to be stirred and heated without adding substrate or auxiliary substances. At a MFR of 200 Nm³/(m³d), a substrate exchange of 4 kilograms per standard cubic metre of methane formed was carried out every one and a half hours.

It emerges from the test results that when applying the process in accordance with the present invention with an exchange rate in which old substrate is exchanged for fresh substrate as a function of the quantity of methane formed and correspondingly, more auxiliary substances such as nutrients and trace elements are added, a bioreactor with different methane formation rates can be operated in a stable manner and in addition, start-stop operation of the bioreactor is possible.

The bioreactor could be operated in a stable manner over the entire time period of the test, without adjusting the pH by measurement of the pH in the reactor and the appropriate addition of base or acid. The pH was determined by sampling from samples of the discharged old substrate and was in a range between pH 7.5 and pH 8.3 throughout the entire time period of the test. In addition, the measured data showed that a methane-enriched gas was obtained as the product gas without further gas preparation, which gas had a very high methane content of at least 95% with carbon dioxide concentrations of approximately 1% and a hydrogen concentration of less than 4%.

Thus, by means of a process in accordance with the present invention, different levels of methane formation rates could be obtained and different downtimes for the methanation process were also tolerated. Because of the fluctuating availability of renewable or cheap power for electrolysis for hydrogen production and also the fluctuating quantity of fermentation gas as the source of carbon dioxide, these facts constitute enormous advantages.

### Example 3

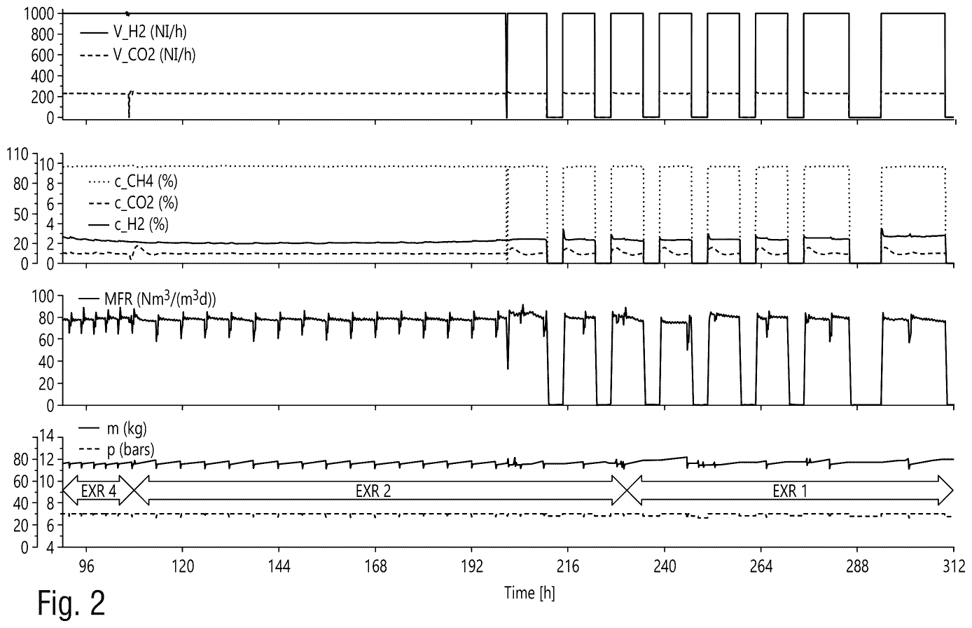

FIG. 2 shows, as a graphical representation, measured values in four diagrams as a function of the test time [h] for the same parameters as for Example 2 with reference to FIG. 1.

The biological methanation was carried out in a bioreactor which contained digested sludge from a municipal sewage treatment plant as the methanation medium, to which individual trace elements and nutrients had been added. The biological methanation was carried out with the addition of methanogenic microorganisms from the order Methanobacteriales, which was added at the start of the test as an inoculum. No methanogenic microorganisms were added during the further course of the test. The production of the methane-enriched gas was carried out in a continuous stirred-tank reactor with a 75 litre reactor content, at a temperature of 65° C. and a pressure of 7 bar.

In contrast to Example 2, the biological methanation was carried out over the entire time period with a constant methane formation rate (MFR) of 80 standard cubic metres of methane per cubic metre of reactor contents per day [Nm³/(m³d)], whereas the exchange rate (EXR) was varied. The exchange with fresh substrate was carried out batchwise, as described in Example 1.

Individual nutrients and trace elements were supplied to the reactor after the substrate exchange via a pump from a reservoir of concentrated stock solutions, respectively over a short time period. The respective quantity was in agreement with the quantity of exchanged substrate and was calculated according to predetermined target values for the individual nutrients and trace elements.

For the first six exchange procedures shown in FIG. 2, substrate was supplied at an exchange rate (EXR) of 4 kg of fresh substrate per standard cubic metre of methane formed [kg/Nm³]. From a test time of 108 hours to a test time of 232 hours, the substrate exchange was carried out at an EXR of 2 kg of fresh substrate per standard cubic metre of methane formed, and afterwards at an EXR of 1 kg of fresh substrate per standard cubic metre of methane formed. The corresponding time intervals for the different EXRs are shown in the bottom diagram.

From a test time of 210 hours, the biological methanation was carried out in a start/stop operation each with 8 hours of gassing and 4 hours of “hot standby”. Because the exchange procedure was triggered each time, as soon as 5% of the reactor volume was to be exchanged at the corresponding methane formation rate, the substrate exchange was carried out at an EXR of 4 kg/Nm³ twice as frequently as with an EXR of 2 kg/Nm³ and four times as frequently as with an EXR of 1 kg/Nm³. At the exchange rates shown of 4 kg/Nm³, 2 kg/Nm³ and 1 kg/Nm³, the corresponding periods to the next substrate exchange were approximately 3 hours, 6 hours or 12 hours. In start/stop operation, the downtimes were also 4 hours each.

As can be seen from the profile of the test, a stable methanation was obtained at the different exchange rates. The gas qualities remained almost constant with a methane content of 95% to 96%, a hydrogen content of 2% to 3% and a carbon dioxide content of approximately 1%. In addition, the pH remained stable in a range between 7.9 and 8.1 without the addition of acids or bases.

Example 3 shows that different quantities of substrate per quantity of methane formed can be exchanged. With the substrate exchange, each time, the same quantity of nutrients, trace elements or optional other auxiliary substances was added. If the fresh substrate for the biological methanation is an organic substrate with an increased viscosity such as sewage sludge or fermentation residue, for example, a different exchange rate can be used as the technical means for influencing the viscosity in the bioreactor.

Because 1.6 litres of water per standard cubic metre is formed during the biological methanation, at a smaller exchange rate, the substrate in the bioreactor is substantially more strongly diluted than at a higher exchange rate. Beginning with a viscosity of approximately 0.03 Pa·s at an EXR of 4 kg/Nm³, in Example 3, the viscosity dropped to a value of approximately 0.003 Pa·s at an EXR of 1 kg/Nm³. In individual cases this can be used as a technical characteristic of the methanation process, for example when a fresh substrate has a relatively high viscosity which would require a high agitator power or would cause a high energy consumption upon agitation. If an organic substrate itself already contains large quantities of essential trace elements and nutrients, it is advantageous to use rather higher exchange rates so that as few as possible, or in the best case no, nutrients and trace elements have to be additionally supplied.

### Example 4

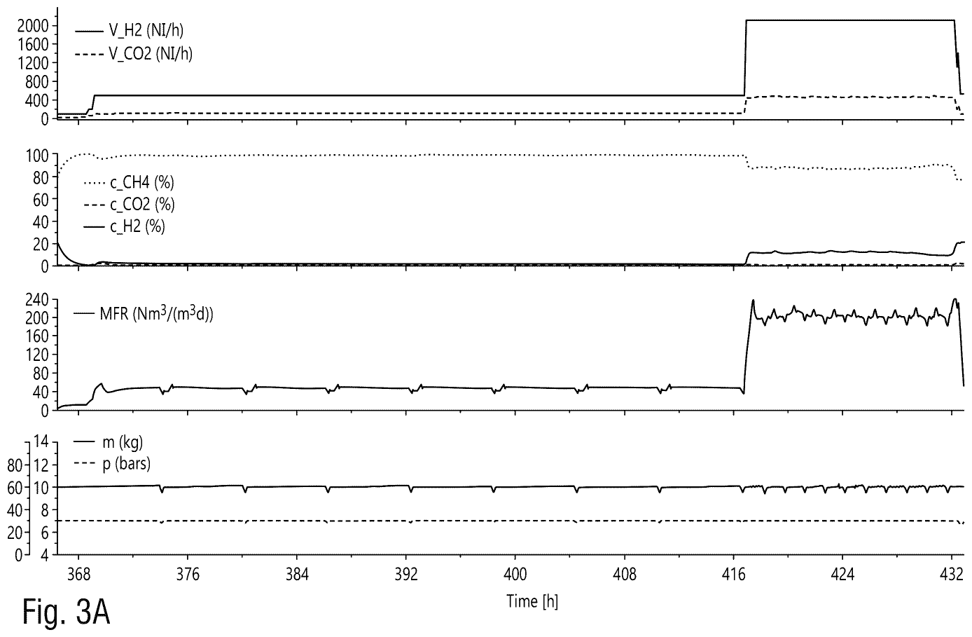

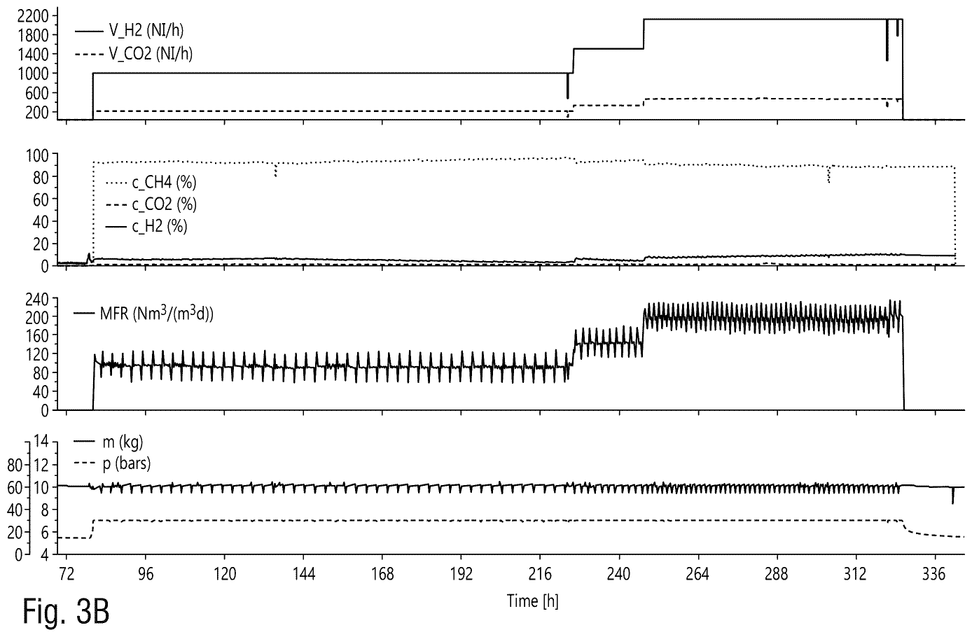

FIGS. 3A and 3B show, as a graphical representation, measured values in four diagrams as a function of the test time [h] for the same parameters as in Example 2 with reference to FIG. 1.

FIGS. 3A and 3B show measured values for the product gas composition and procedural parameters when implementing an embodiment of the process in accordance with the present invention with a synthetic culture medium at a constant exchange rate and different methane formation rates, wherein the composition of the synthetic culture medium in the test forming the basis of the measured values of FIG. 3A differ from the composition of the synthetic culture medium in the test forming the basis of the measured values of FIG. 3B.

The biological methanation was carried out in a bioreactor with a gassing agitator, which contained a synthetic culture medium as the methanation medium, as used in the prior art for hydrogenotrophic methanogenic archaea (see, for example, WO 2008/094282 A1 or WO 2012/110257 A1). The medium was inoculated with a methanogenic strain from the genus Methanothermobacter.The production of the methane-enriched gas was carried out in a continuous stirred-tank reactor with a 60 litre reactor content, at a temperature of 65° C. and a pressure of 7 bar.

The biological methanation was carried out over the entire time period at an exchange rate (EXR) of 4 kilograms of fresh substrate per standard cubic metre of methane formed [kg/Nm³]. The exchange of fresh substrate was carried out batchwise as described with reference to Example 1. A mineral culture medium was used as the fresh substrate. Nutrients and trace elements from a concentrated stock solution were respectively supplied to the reactor over a short time period as auxiliary substances. The respective quantity was in agreement with the quantity of exchanged substrate and was calculated according to specified target values for the individual nutrients and trace elements. Sodium hydroxide was repeatedly used as a further auxiliary substance in order to stabilise the pH, as well as defoaming agents if necessary. The addition of base and defoaming agent was carried out independently of the change of substrate.

In the test shown in FIG. 3A, after appropriate adjustment of the volumetric educt gas flows, the bioreactor was operated for 2 days at a methane formation rate of 50 Nm³/(m³d). Subsequently, the methane formation rate was increased to 200 Nm³/(m³d) and methane-enriched gas was produced for somewhat more than one day. At a MFR of 50 Nm³/(m³d), an exchange of fresh substrate of 5% of the reactor target mass was carried out every 6 hours; at a MFR of 200 Nm³/(m³d), an exchange of the same quantity of fresh substrate was carried out every 1.5 hours. In the test shown in FIG. 3B, the bioreactor was operated for 10 days with increasing methane formation rates of 100 Nm³/(m³d), subsequently 145 Nm³/(m³d) and finally 200 Nm³/(m³d). The exchange intervals correspondingly had a duration of 3 hours, approximately 2 hours and 1.5 hours. At all of the methane formation rates, the product gas had a constant proportion of approximately 1% carbon dioxide. While the methane content at a MFR of 50 Nm³/(m³d) was approximately 97% and the hydrogen content was approximately 2%, at a MFR of 145 Nm³/(m³d), the methane content was still 93.5% and the hydrogen content was approximately 5.5%; finally, at a MFR of 200 Nm³/(m³d), the methane content was 90% and the hydrogen content was 9% of the product gas.

Example 4 shows that when an embodiment of the process in accordance with the present invention is implemented even when using pure cultures in synthetic media at an exchange rate which is a function of the quantity of methane formed and different methane formation rate levels, a stable biological methanation is obtained. A methane-enriched gas with a very high proportion of methane is obtained which can be purified by a gas preparation with hydrogen separation into a biomethane which can be fed into the grid.

### Comparative Example

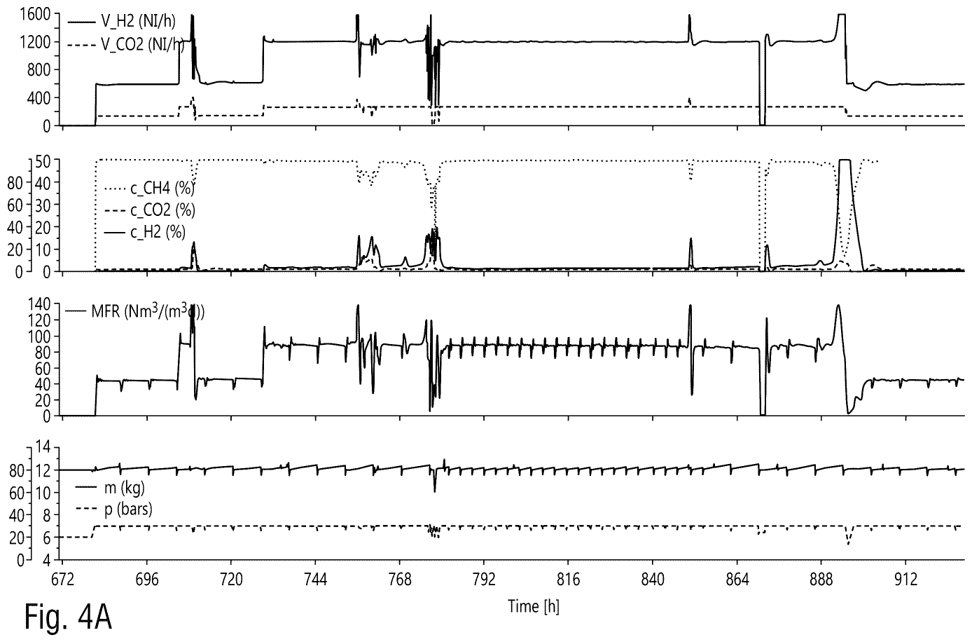

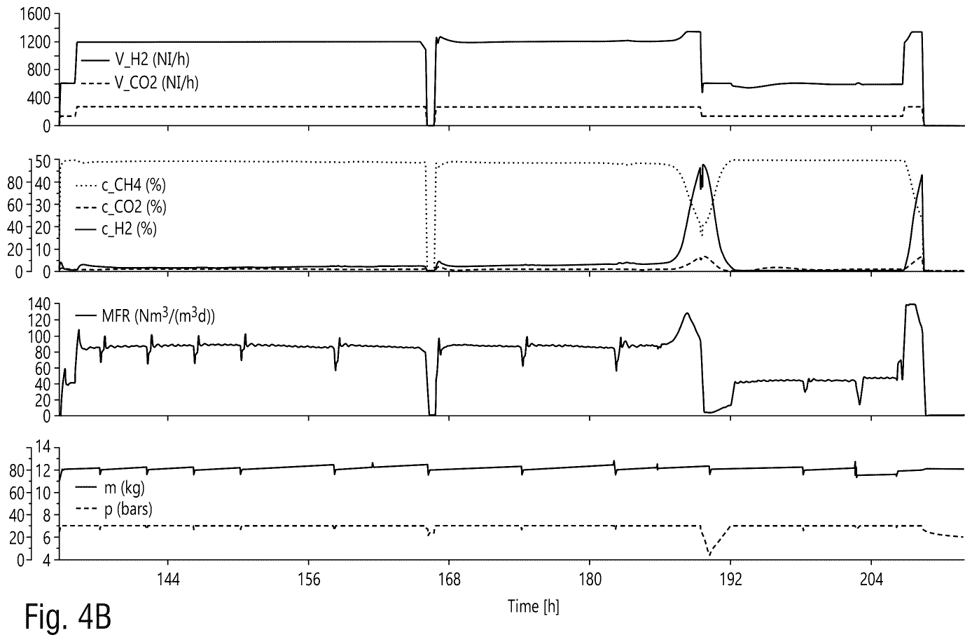

FIGS. 4A and 4B show, as a graphical representation, measured values in four diagrams as a function of the test time [h] for the same parameters as in Example 2 with reference to FIG. 1.

In this comparative example, the substrate exchange was not carried out as a function of the quantity of methane formed, but rather, a specific quantity of substrate and of auxiliary substances was replaced as a function of time.

The biological methanation was carried out in a bioreactor which contained digested sludge from a municipal sewage treatment plant as the methanation medium, to which individual trace elements and nutrients had been added. The biological methanation was carried out without the addition of a methanogenic microorganism. The production of the methane-enriched gas was carried out in a continuous stirred-tank reactor with a 80 litre reactor content, at a temperature of 65° C. and a pressure of 7 bar.

As can be seen in FIG. 4A, at the beginning, at a test time of 680 hours in the bioreactor, the addition of the volumetric educt gas flows of carbon dioxide and hydrogen were set in a manner such that over a time period of 24 hours, a methane formation rate (MFR) of 40 standard cubic metres of methane per cubic metre of reactor content per day [Nm³/(m³d)] was obtained with a gas quality of 99% CH₄, 1% CO₂ and 0% H₂. Approximately every 8 hours, a substrate exchange of 5% of the reactor volume was carried out, corresponding to an exchange rate (EXR) of 4 kilograms of fresh substrate per standard cubic metre of methane formed [kg/Nm³].

Following this, the methane formation rate was to be increased without at the same time increasing the number of substrate exchanges. To this end, after the third substrate exchange, the educt gas quantity was increased so that a MFR of 88 Nm³/(m³d) was obtained. Starting from a MFR of 88 Nm³/(m³d), after 3 hours, the educt gas supply was increased in a manner such that a MFR of 120 Nm³/(m³d) should have been produced. However, this was not possible, because the gas quality deteriorated significantly to values of 76% CH₄, but 10% CO₂ and 14% H₂.

From a test time of 710 hours, the bioreactor was operated for 18 hours more with a MFR of 40 Nm³/(m³d). From a test time of 729 hours, the educt gas quantity was raised again in a manner such that a MFR of 88 Nm³/(m³d) was produced. However, the exchange times were not adjusted in correspondence with the increased methane production, but a substrate exchange was still carried out only every 8 hours. Since after 26 hours, the educt gas addition was increased significantly in order to obtain a MFR of 120 Nm³/(m³d) at full conversion, a severe decline in the gas quality occurred again, so that the increase in the educt gas supply was reduced again to the quantity which corresponded to a MFR of 80 Nm³/(m³d).

Two further exchanges of 4 kg of fresh substrate, each time after 8 hours, was then carried out, as originally set for a MFR of 40 Nm³/(m³d). From a test time of 774 hours, the product gas quality dropped rapidly without having increased the educt gas supply. A probable explanation is that the supply of nutrients and trace elements was no longer sufficient, because methanation had taken place over a longer time period at a higher methane formation rate. After a substrate exchange of 20 kg, which corresponded to a quarter of the reactor content, 4 kg of fresh substrate was exchanged every 3 h 20 min and the appropriate nutrients and trace elements were added. Converted to the corresponding quantity of methane formed, this is equivalent to the quantity which was added at a MFR of 40 Nm³/(m³d) for the smaller number of exchange cycles. At this higher addition of fresh substrate, the methanation could be continued at a MFR of 88 Nm³/(m³d) with a very good gas quality of 99% CH₄, 1% CO₂ and 0% H₂.

After 68 h, a further attempt was made to increase the gas supply in a manner such that a MFR of 120 Nm³/(m³d) could be obtained. However, this again resulted in a decline in the gas quality. From a test time of 854 h, the number of substrate exchanges at a MFR of 88 Nm³/(m³d) was again reduced to how they were originally carried out at a MFR of 40 Nm³/(m³d). At test time 870 h, there was a brief interruption in the methanation because of a technical fault at the gas station. In total, the biological methanation functioned at the smaller number of exchanges at a MFR of 88 Nm³/(m³d) over a time period of approximately 38 hours. From the test time of 890 hours, however, there was a very severe decline in both the gas quality as well as in the quantity of methane formed. By reducing the gas supply, the methanation could be continued with good gas quality at a MFR of 40 Nm³/(m³d) with an exchange of 4 kg of substrate every 8 hours.

In a further test, the measured values which are shown in FIG. 4B are a graphical representation in each of four diagrams as a function of the test time [h] for the same parameters as described in Example 2 with reference to FIG. 1, again, the substrate exchange was not carried out as a function of the quantity of methane formed, but rather, a specific quantity of substrate and auxiliary substances was respectively exchanged as a function of time.

The biological methanation was carried out in a bioreactor which contained digested sludge from a municipal sewage treatment plant as the methanation medium, to which individual trace elements and nutrients had been added. The biological methanation was carried out without the addition of a methanogenic microorganism. The production of the methane-enriched gas was carried out in a continuous stirred-tank reactor with an 80 litre reactor content, at a temperature of 65° C. and a pressure of 7 bar.

From a test time of 136 hours, the educt gas supply of hydrogen and carbon dioxide into the bioreactor was adjusted in a manner which resulted in a MFR of approximately 87 Nm³/(m³d) with a very good gas quality of 98% CH₄, 1% CO₂ and 1% H₂. A substrate exchange of 5% of the reactor volume approximately every 4 hours was carried out, corresponding to an exchange rate (EXR) of 4 kilograms of fresh substrate per standard cubic metre of methane formed.

From a test time of 150 hours, 4 kilograms of fresh substrate was exchanged every 8 hours and the appropriate quantity of nutrients and trace elements was added. At test time 166 hours, there was a technical fault at the gas station, so that there was no gas supply for one hour and in addition and accordingly, no product gas entered the line.

After the 4^{th} substrate change which was undertaken in the cycle of 8 hours per substrate exchange, from a test time of 182 hours, the gas quality deteriorated significantly. From a test time of 187 hours, the gas quality declined sharply, so that the educt gas supply was cut back to a quantity which should have resulted in a MFR of 40 Nm³/(m³d) in the case of full conversion of the gases. Under these conditions, with a substrate exchange every 8 hours, a biomethane with a very good gas quality (99% CH₄, 1% CO₂, less than 0.2% H₂) could be obtained. When the supply of educt gases was increased again at test time 207 hours, the gas quality declined again.

Because the pH was constant in a range of 7.0 to 7.3 without large fluctuations over the entire time period of the tests described in FIGS. 4A and 4B, a varying pH could not be the reason for the deterioration in the biological methanation.

Rather, the comparative example shows that a time-dependent exchange of a specific quantity of substrate as well as nutrients and trace elements delivers significantly poorer results for the biological methanation than the substrate exchange in accordance with the invention, which is carried out as a function of the quantity of methane formed.

1. A process for the production of a methane-enriched gas, comprising the steps of:
a) providing a bioreactor having:
at least one inlet for supplying hydrogen and carbon dioxide to the bioreactor,
at least one inlet for supplying fresh substrate to the bioreactor,
at least one outlet for removing old substrate from the bioreactor, and
at least one outlet for removing methane-enriched gas formed in the bioreactor,
b) removing the methane-enriched gas formed in the bioreactor from the bioreactor,
c) determining a quantity of methane formed in the bioreactor in a predetermined time period,
d) removing old substrate from the bioreactor, and
e) supplying fresh substrate to the bioreactor,
wherein a quantity of fresh substrate supplied to the bioreactor is a function of the quantity of methane formed in the bioreactor in the predetermined time period.

2. The process as claimed in claim 1, wherein the quantity of fresh substrate, m_{fresh substrate}, supplied to the bioreactor is the function of the quantity of methane, Vₘₑₜₕₐₙₑ, formed in the bioreactor in the predetermined time period and of an exchange rate, EXR, and satisfies an equation:
m_{fresh substrate}=Vₘₑₜₕₐₙₑ·EXR
wherein the exchange rate, EXR, satisfies a condition EXR>0.

3. The process according to claim 2, wherein the quantity of fresh substrate, m_{fresh substrate} [kg], supplied to the bioreactor is the function of the quantity of methane, Vₘₑₜₕₐₙₑ [Nm³], formed in the bioreactor in the predetermined time period and of the exchange rate, EXR [kg/Nm³], and satisfies the equation:
m_{fresh} substrate [kg]=Vₘₑₜₕₐₙₑ [Nm³]·EXR [kg/Nm³]
wherein the exchange rate, EXR, satisfies the condition 0.01 kg/Nm³≤EXR≤10 kg/Nm³.

4. The process according to claim 3, wherein the exchange rate, EXR, satisfies the condition 1 kg/Nm³≤EXR≤6 kg/Nm³.

5. The process according to claim 1, wherein the fresh substrate supplied in the step e) is a biological substrate with an increased viscosity or a culture medium.

6. The process according to claim 5, wherein the biological substrate with the increased viscosity is fermentation residue from a biogas plant or sewage sludge.

7. The process according to claim 5, wherein the culture medium contains one or more of: microorganisms, nutrients, and trace elements.

8. The process according to claim 1, wherein after the step d) and before, together with or after the step e), step f) of supplying one or more auxiliary substances to the bioreactor is carried out, wherein the one or more auxiliary substances are nutrients, trace elements, defoaming agents, acids, bases or mixtures thereof.

9. The process according to claim 1, wherein the steps d) and e) are carried out continuously.

10. The process according to claim 9, wherein the removal of old substrate from the bioreactor in step d) and the supply of fresh substrate to the bioreactor in step e) are carried out in a manner such that a total quantity of substrate present in the bioreactor remains substantially constant, wherein the total quantity of substrate present in the bioreactor corresponds to a predetermined target quantity of substrate.

11. The process according to claim 1, wherein the steps d) and e) are carried out in a batchwise manner.

12. The process according to claim 11, wherein the quantity, m_{fresh substrate} [kg], of fresh substrate supplied to the bioreactor per batch is a function of a predetermined target quantity of substrate, m_{target substrate} [kg], present in the bioreactor and satisfies a condition:
0.01 m_{target substrate}≤m_{fresh substrate}≤0.1 m_{target substrate}.

13. The process according to claim 1, wherein the determination of the quantity of methane, Vₘₑₜₕₐₙₑ, formed in the bioreactor in the predetermined time period in step c) is carried out by determining a quantity of water, V_{water}, formed in the bioreactor in the predetermined time period, wherein a quantity of 1 Nm³ of methane which is formed corresponds to a quantity of 1.6 L of water.

14. A plant for carrying out the process according to claim 1, wherein the plant has at least one bioreactor, wherein the bioreactor has:
at least one inlet for supplying hydrogen and carbon dioxide to the bioreactor,
at least one inlet for supplying fresh substrate to the bioreactor,
at least one outlet for removing old substrate from the bioreactor, and
at least one outlet for removing methane-enriched gas formed in the bioreactor,
wherein the plant furthermore comprises:
a device for determining the quantity of methane formed in the bioreactor in a predetermined time period, and
a computer unit, wherein the computer unit is designed and configured to calculate the quantity of fresh substrate to be supplied to the bioreactor as a function of the quantity of methane formed in the bioreactor in the predetermined time period.

15. The plant according to claim 20, wherein the computer unit is designed and configured to calculate the quantity of fresh substrate, m_{fresh substrate}, to be supplied to the bioreactor as the function of the quantity of methane, Vₘₑₜₕₐₙₑ, formed in the bioreactor and an exchange rate, EXR, in accordance with an equation:
m_{fresh substrate}=Vₘₑₜₕₐₙₑ·EXR
wherein the exchange rate, EXR, satisfies a condition EXR>0.

16. The plant according to claim 15, wherein the computer unit is designed and configured to calculate the quantity of fresh substrate, m_{fresh substrate} [kg], to be supplied to the bioreactor as the function of the quantity of methane, Vₘₑₜₕₐₙₑ [Nm³], formed in the bioreactor and the exchange rate, EXR [kg/Nm³], in accordance with the equation:
m_{fresh substrate} [kg]=Vₘₑₜₕₐₙₑ [Nm³]·EXR [kg/Nm³],
wherein the exchange rate, EXR, satisfies the condition 0.01 kg/Nm³≤EXR≤10 kg/Nm³.

17. The plant according to claim 20, wherein the plant furthermore has at least one substrate pump, wherein the at least one substrate pump is designed and configured to deliver fresh substrate to the at least one inlet for supplying fresh substrate to the bioreactor and/or to remove old substrate from the bioreactor.

18. The plant according to claim 17, wherein the computer unit is designed and configured to control the at least one substrate pump, wherein the control of the at least one substrate pump is carried out in a manner such that the fresh substrate is supplied to the bioreactor in accordance with an equation:
m_{fresh substrate}=Vₘₑₜₕₐₙₑ·EXR.

19. The plant according to claim 17, wherein the computer unit is designed and configured to control the at least one substrate pump, wherein the control of the at least one substrate pump is carried out in a manner such that the fresh substrate is continuously supplied to the bioreactor in a quantity, m_{fresh substrate} [kg], such that a total quantity, m_{total substrate} [kg], of substrate present in the bioreactor remains substantially constant, wherein the total quantity of substrate present in the bioreactor corresponds to a predetermined target quantity of substrate, m_{target substrate} [kg].

20. A plant for the production of a methane-enriched gas, the plant comprising:
at least one bioreactor, wherein the bioreactor has:
at least one inlet for supplying hydrogen and carbon dioxide to the bioreactor,
at least one inlet for supplying fresh substrate to the bioreactor,
at least one outlet for removing old substrate from the bioreactor, and
at least one outlet for removing methane-enriched gas formed in the bioreactor,
wherein the plant furthermore comprises:
a device for determining a quantity of methane formed in the bioreactor in a predetermined time period, and
a computer unit, wherein the computer unit is designed and configured to calculate a quantity of fresh substrate to be supplied to the bioreactor as a function of the quantity of methane formed in the bioreactor in the predetermined time period.

21. The plant according to claim 16, wherein the exchange rate, EXR, satisfies the condition 1 kg/Nm³≤EXR≤6 kg/Nm³.
